(19) United States
(12) Patent Application Publication
(10) Pub. No.: US 2024/0342344 A1
(43) Pub. Date: Oct. 17, 2024

(54) Conformal Shaped Biological Construct

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 18/632,805

(22) Filed: Apr. 11, 2024

Related U.S. Application Data
(60) Provisional application No. 63/495,861, filed on Apr. 13, 2023.

(51) Int. Cl. A61L 27/58; A61L 27/24

(52) U.S. Cl. CPC A61L 27/58; A61L 27/24; A61L 2430/34

(57) ABSTRACT

A biological construct that conforms for fixation to a torn labrum, meniscus or tendon. The device includes a resilient triangularly or tent shaped construct that conforms to the anatomy to be treated. The construct includes a first a first surface, a second substrate surface and a third collagen surface.

This application claims priority to U.S. Provisional Application 63/495, 861 filed Apr. 13, 2023.

### FIELD OF THE INVENTIONS

The inventions described below relate to the field of biological constructs used in arthroscopic surgery.

### BACKGROUND OF THE INVENTIONS

Conformal biological constructs used during arthroscopic procedures can improve integration to ensure proper healing and long-term stability within a patient. Conformal biological constructs offer advantages in terms of structural stability, surface area coverage and increases cellular interactions. This provides advantages in coverage of the torn tissue in order to promote enhanced construct attachment and facilitate and improve regeneration of tissue healing and function. The various structural shapes of the construct can maximize the interaction between the biological construct and the torn tissue.

### SUMMARY

The devices and methods described below provide for an improved biological construct that conforms for fixation to a torn labrum, meniscus or tendon. The device comprises a resilient triangularly or tent shaped implant that conforms to the anatomy to be treated. When placed over the tear, the conformal shape allows for improved cell attachment and healing, thereby increasing the construct efficiency.

The shaped construct includes a spring frame that includes a substrate backing or scaffold surface and a porous collagen surface or layer. The construct is triangular or tent shaped and includes an apex. The spring frame is collapsible so that it can be loaded within a delivery tube and delivered to a desired repair site. Once at the desired repair site, the spring can be open or expanded to the original triangular or tent shape and placed over a torn labrum or meniscus and secured within a patient.

The shape of the biological construct boosts adherence and promotes maximum cell growth because it completely encases the torn area. The construct provides improved ability to assimilate into the host environment and defined geometry adapts well to regeneration and boosts adherence and cell growth. The second and third surfaces are made of a non-porous and moisture resistant biomaterial material.

### BRIEF DESCRIPTION OF THE DRAWINGS

FIG. 1 illustrates the glenoid labrum of a shoulder with a labral tear.

FIG. 2 illustrates a meniscus of a knee with a lateral meniscal tear.

FIG. 3 is an exploded view of a conformal shaped biological construct.

FIG. 4 is a view of the conformal shaped biological construct positioned over a labral tear.

FIG. 5 is a view of a conformal shaped biological construct positioned over a meniscal tear.

### DETAILED DESCRIPTION OF THE INVENTIONS

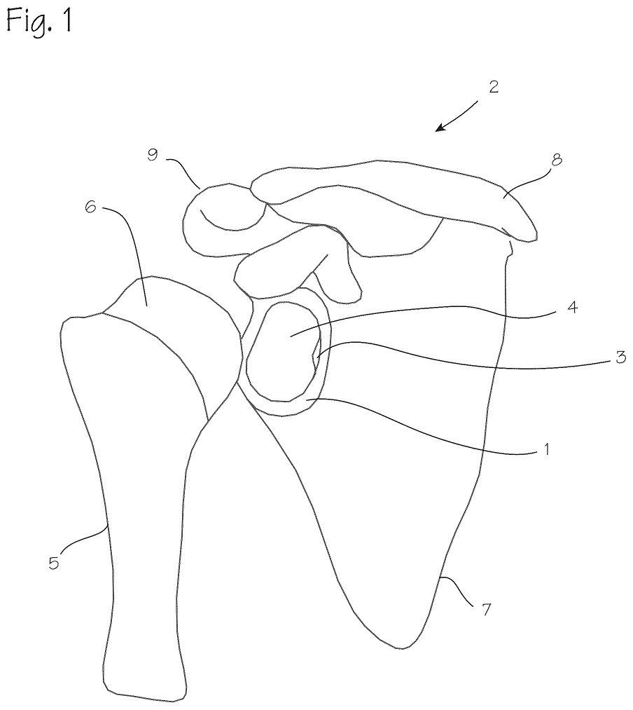

FIG. 1 illustrates the labrum, particularly the glenoid labrum 1 of the shoulder 2 of a patient showing a labral tear 3 of the glenoid 4 and surrounding anatomy. The humerus 5 is the long bone in the upper arm that extends from the shoulder to the elbow and the humeral head 6 is the rounded structure at the proximal end of the humerus. The humeral head articulates with the glenoid of the scapula or shoulder blade 7 to form the shoulder joint. The clavicle or collarbone 8, connects the sternum to the scapula. The acromion 9 is a flat, triangular shaped bone that articulates with the clavicle to form the acromioclavicular joint.

The labrum 1 is the cup shaped rim of cartilage that lines and reinforces the ball-and-socket joint of the shoulder. The labrum is the attachment site for the shoulder ligaments and supports the ball-and-socket joint as well as the rotator cuff tendons and muscles. The labrum in cross section is generally triangular. A base is fixed to the circumference of the cavity and the free edge is thin and sharp. Below the equatorial pole of the glenoid 4, the labrum becomes more rounded and smaller compared to superiorly where it is more triangular in shape.

The shoulder labrum forms a ring-shaped structure that surrounds the socket of the glenoid. It is thicker in the superior (top) portion and thinner towards the inferior (bottom) portion. The average thickness of the labrum is between 2 mm to 4 mm. A labrum is also contained within the hip. Hip labrum is also circular or oval shaped structure that attaches to the rim of the socket of the pelvis. The hip labrum is smaller than the shoulder labrum and forms rim-like structure around the hip socket (acetabulum). The average thickness of the hip labrum is between 1 mm to 2 mm. The height of the labrum is the thickness of depth of the labral tissue from its attachment point on the rim of the joint socket (glenoid in the shoulder or the acetabulum in the hip) to its free edge. The average height of the shoulder labrum is between 2 mm to 4 mm and the average height of the hip labrum is between 1 mm to 2 mm. Tearing of the labrum can occur from either acute trauma or repetitive shoulder motion. Acute trauma may be from dislocation of the shoulder, direct blows to the shoulder or other accidents. Major detachment requires surgery. However, arthroscopic procedures can be used for minor attachments.

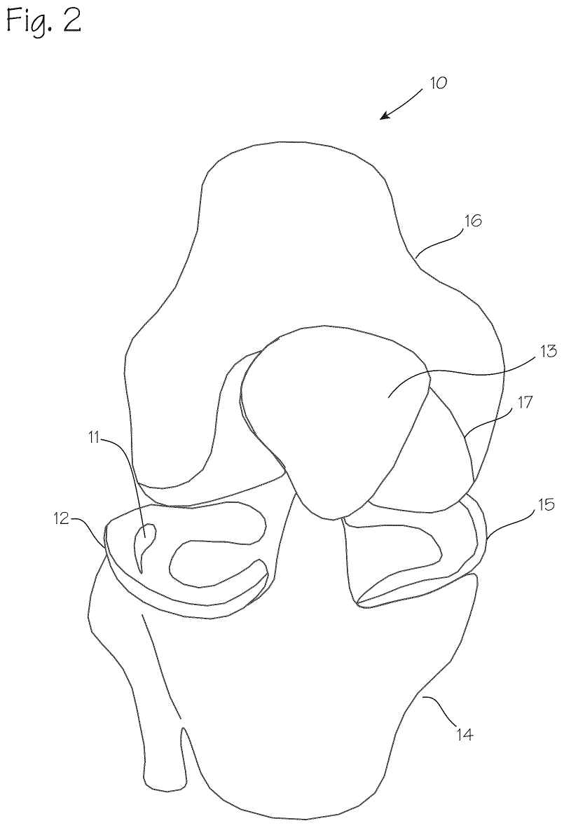

FIG. 2 illustrates the meniscus within a knee joint 10 of a patient showing a meniscal tear 11 within the lateral meniscus 12 and surrounding anatomy. The meniscus is contained within the patella or kneecap 13, which is the triangular shaped bone located in the front of the knee joint. Meniscus is a C-shaped structure that conforms to the shape of the tibia 14. The outer edge of each meniscus is thicker and more firmly attached to the knee joint while the inner edge is thinner. The knee joint further includes a medial meniscus 15 that is crescent shaped. The femur or thigh bone 16 includes femoral condyles 17 situated at the distal end of the femur. The average thickness of the lateral meniscus is between 2 mm to 4 mm. The height of the lateral meniscus is the vertical dimension of the meniscus, from the superior (upper) surface to the anterior (lower) surface. The average height of the lateral meniscus is between 9 mm and 11 mm.

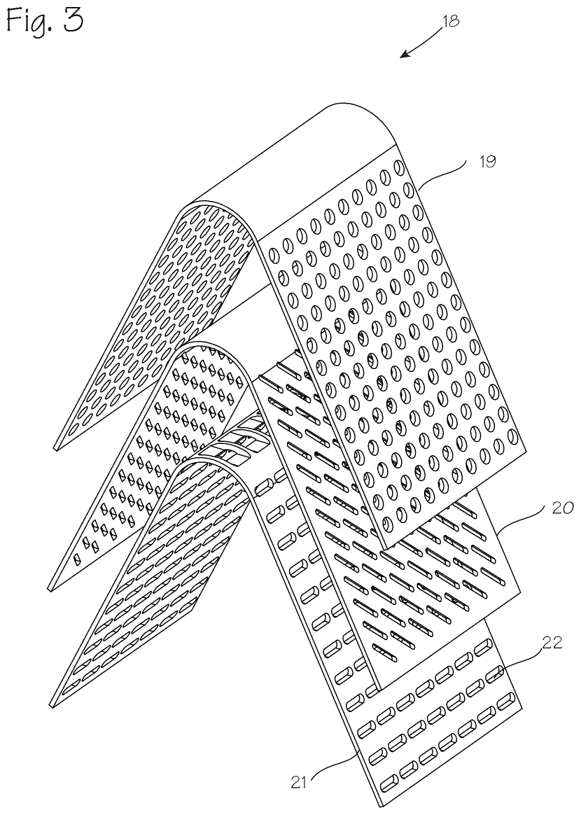

FIG. 3 is an exploded view of a conformal shaped biological construct 18. The biological construct has a generally triangular or tent shape that conforms to the shape of the labrum or meniscus. The biological patch includes a first resilient spring surface 19, a second substrate surface 20 and a third collage surface 21. The third collage surface includes collagen fibers 22 formed in an aligned orientation where the collagen fibers are aligned in the same direction, running parallel to each other. The fibers are thin, elongate structures, arranged closely together in a uniform pattern. The parallel alignment or orientation reinforces the structure of the labrum or meniscus and provides resistance to forces acting along the direction of the fibers. The aligned or parallel orientation mimics the connective tissue of labrum or meniscus and assists in supporting and maintaining tissue to support healing. The second substrate surface is contained between the first resilient spring surface and the third collagen surface. When positioned within a patient, the collagen surface is placed over the torn tissue. Each surface is triangular or tent shaped with a rounded apex that is tapered to fit into small spaces to provide a more anatomical fit to the labrum or meniscus. The apex is shaped to conform to the shape of either a labrum or meniscus. The apex is arranged in an angle of 45 degrees or less. The shaped construct allows the construct to cover the damaged area effectively and provide stability to the repair joint. The apex of the construct is rounded and creates a construct opening of between 1 mm to 4 mm to fit securely over a torn labrum or meniscus. The sides of the construct are dimensioned to be positioned over the surface of the torn labrum or meniscus to provide stability and support when the construct is positioned over the torn tissue. The height of the construct is between 1 mm to 11 mm to cover the entire height of a torn labrum or meniscus.

The first spring surface 20 is comprised of a spring material. Suitable materials include Polyglycolic Acid, Poly-Lactic Acid, Poly-L-Lactic Acid (PGA, PLLA), Polyglygolic Acid (PGA), Poliglecaprolactone (PLCL), Polycaprolactone (PCL), Polydioxinone (PDS), Polyglyconate, ATPE or other similar material. The spring surface is between 0.1 to 5 mm thick. The spring surface can be of any ribbed or meshed geometry. The substrate surface is the scaffold or matrix that mimics the natural extracellular environment found in living tissues. The substate surface 21 is made of an absorbable material with spring memory properties. Suitable materials include polycaprolactone, Poly (1-lactide-co-ϵ-caprolactone), copolymers or polydioxanone. The substrate surface can be between 0.1 mm to 5 mm thick and can have a porosity of between 50 microns and 500 microns. The substrate surface can be formed by electrospinning or can be woven. The third collagen surface includes collage cells intended for tissue regeneration that can be seeded onto or into the substrate surface. The collagen surface 22 can be formed of any bovine, porcine or synthetic recombinant collagen having a porosity between 50 microns and 500 microns. The biological construct or implant may contain non-absorbable ultra-high-molecular-weight polyethylene (UHMWPE) nylon, polyester, fibers, or superelastic nickel titanium alloy wires. The collage surface can be formed by electrospinning, using 3D printing technology, injection molded or thermally formed.

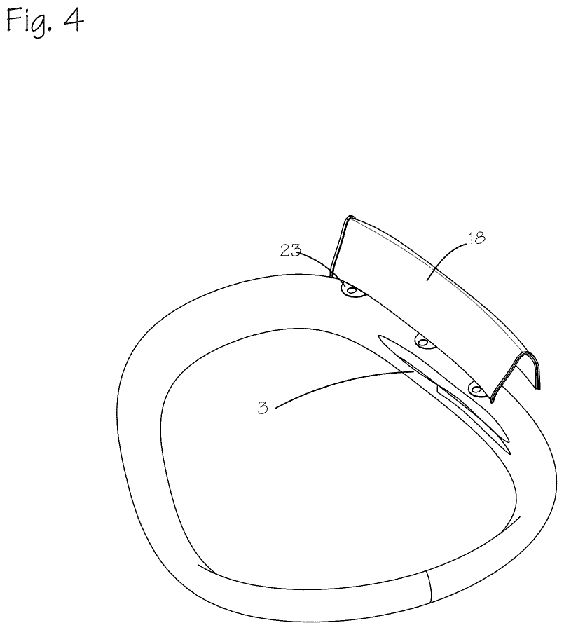

FIG. 4 is a view of the conformal shaped biological construct positioned over a labral tear. The construct is positioned over the torn labrum located in the shoulder. In the delivered position, the construct straddles the torn labrum and conforms to the shape of the labrum to snuggly cover the tear. The construct can optionally be secured within the patient via a loop fastener or grommet 23. The grommet can be used to secure anchors or staples to the bone or the loop fastener can be used to suture the construct directly to the tendon.

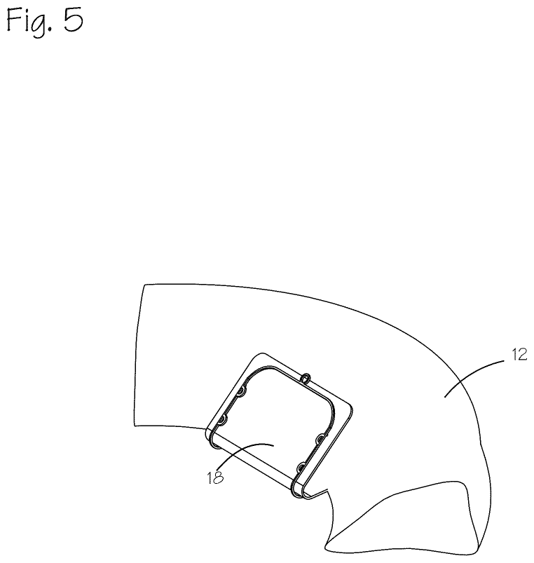

FIG. 5 is a view of a conformal shaped biological construct positioned over a meniscal tear. The construct is positioned over the torn meniscus located in the knee. In the delivered position, the construct straddles the torn meniscus and conforms to the shape of the meniscus to snuggly cover the tear. The construct can optionally be secured within the patient via a loop fastener or grommet.

In use, incisions are made to create portal access to a torn shoulder or hip labrum or meniscus in a patient. A delivery cannula or tube is inserted into an incision site to allow access of the construct to a desired repair site. The construct is collapsed or rolled within the delivery cannula so that the biological construct is constrained within the cannula. The biological construct is introduced through the cannula and advanced through a cannula via a flexible or malleable drive rod into the surgical workspace. The construct is advanced through the cannula until it is positioned over the torn labrum or meniscus. When the construct is positioned over the torn tissue, the construct springs into its open or preformed state resulting in a triangular construct within the patient. The construct is held in place by the introducer. The construct is secured to the labrum or meniscus so that the apex of the construct is positioned over the torn tissue and the sides of the construct conform to the torn tissue. The construct includes a plurality of fastener loops or grommets that accept sutures, fixation staples or tacks. The sutures can be implanted into bone around to labrum or alternatively sutures can be passed through the labral tissue to reattach it to the bone to anchor the construct over the tear and then remove the introducer. The sutures can be tensioned to restore the labrum to its anatomical position and provide stability to the joint.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A biological construct for treatment of a labral tear on a labrum comprising:
a first surface comprising a spring frame;
a second surface comprising a substrate made of a biocompatible material and secured to the first surface; and
a third collage surface comprising a plurality of collagen fibers formed in an aligned orientation, the third collage surface secured to the second surface;
wherein the first, second and third surfaces are triangular or tent shaped and have an apex sized and dimensioned to conform to the shape and size of the labral tear.

2. The biological construct of claim 1 further comprising:
at least one grommet or fastener loop on the first surface for attachment to the labrum.

3. The biological construct of claim 1 further including a construct opening between 1 mm to 4 mm.

4. The biological construct of claim 1 wherein:
the apex is 45 degrees or less.

5. The biological construct of claim 1 wherein:
the aligned collagen fibers are formed in a parallel orientation where each collagen fiber is parallel to each other.

6. The biological construct of claim 1 wherein:
the spring surface is selected from the group consisting of Polyglycolic Acid, Poly-Lactic Acid, Poly-L-Lactic Acid, Polyglygolic Acid, Poliglecaprolactone, Polycaprolactone Polydioxinone, Polyglyconate and ATPE.

7. The biological construct of claim 1 wherein:
the substrate surface is selected from the group consisting of polycaprolactone, Poly (1-lactide-co-ϵ-caprolactone), copolymers or polydioxanone.

8. The biological construct of claim 1 wherein:
the substrate surface is between 0.1 to 5 mm thick.

9. The biological construct of claim 1 wherein:
the substrate surface has a porosity between 50 microns and 500 microns.

10. The biological construct of claim 1 wherein:
the collagen surface is made of a collagen selected from the group consisting of bovine, porcine or synthetic recombinant collagen.

11. The biological construct of claim 1 wherein:
the collage surface has a has a porosity between 50 microns and 500 microns.

12. The biological construct of claim 1 wherein:
the substrate surface is formed by electrospinning.

13. A method of inserting a biological construct through a cannula to treat a labral tear within a patient's body comprising the steps of:
providing a triangular biological construct in a first collapsed configuration, said construct comprising:
a first surface comprising a spring frame;
a second surface comprising a substrate made of a biocompatible material and secured to the first surface; and
a third surface comprising a plurality of aligned collagen layers formed in an aligned orientation and secured to the second surface; wherein the first, second and third surfaces are triangular or tent shaped and have an apex sized and dimensioned to conform to the shape and size of the labral tear;
advancing the biological construct via a drive device through the cannula to the torn labrum;
opening the construct into a second, open configuration and positioning the biological construct over the torn labrum and aligning the biological construct so that the apex of the construct is positioned over the labral tear;
securing the biological construct over the labral tear to prevent displacement of the biological construct; and
removing the delivery device.
